(19) United States
(12) Patent Application Publication
(10) Pub. No.: US 2014/0303657 A1
(43) Pub. Date: Oct. 9, 2014

(54) VASCULAR ANASTOMOSIS DEVICE USING A SELF-EXPANDABLE MATERIAL OR BODY, AND ANASTOMOSIS METHOD FOR BLOOD VESSELS USING SAME

(71) Applicant: THE ASAN FOUNDATION, Seoul (KR)

(72) Inventors: Song Cheol Kim, Seoul (KR); Ha Na Park, Seoul (KR); Si Nae Park, Seoul (KR)

(73) Assignee: THE ASAN FOUNDATION, Seoul (KR)

(21) Appl. No.: 14/355,831

(22) PCT Filed: Nov. 1, 2012
(86) PCT No.: PCT/KR2012/009131

§ 371 Date: May 1, 2014

(30) Foreign Application Priority Data
Nov. 1, 2011 (KR) 10-2011-0112920

(51) Int. Cl. A61B 17/11

(52) U.S. Cl. CPC A61B 17/11; A61B 2017/1107
USPC 606/155

(57) ABSTRACT

The present invention relates to an anastomosis device for blood vessels using a self-expandable material or body, and to an anastomosis method for blood vessels using same, and more particularly, to an anastomosis device for blood vessels using a self-expandable material or body, which is self-expandable in response to external stimulation and which is removable after a procedure, and to an anastomosis method for blood vessels using same.

This application is a national phase application under 35 U.S.C. §371 of International Application Serial No. PCT/KR2012/009131 filed on Nov. 1, 2012, and claims the priority under 35 U.S.C. §119 to Korean Patent Application No. 10-2011-0112920, filed on Nov. 1, 2011 which are hereby expressly incorporated by reference in their entirety for all purposes.

### TECHNICAL FIELD

The present invention relates to a vascular anastomosis device using a self-expandable material or body and a method of vascular anastomosis using the same, and more particularly, to a vascular anastomosis device using a material or a body which has self-expandability in response to an external stimulus and is removable after a procedure, and a method of vascular anastomosis using the same.

### BACKGROUND OF THE INVENTION

A vascular anastomosis, especially a microvascular anastomosis, is being applied to various types of operations, and is an essential operation method for, in particular, plastic surgery or organ transplantation such as kidney transplantation and liver transplantation. Currently, due to lack of exercise, westernized eating habits, and the like, so-called vascular occlusive diseases caused by narrowed or almost occluded blood vessels have been increasing year by year. As a method of treating such occluded blood vessels or almost occluded blood vessels, a surgical method is often employed. A method that is usually employed as such a surgical method is a method in which a part of a blood vessel causing a problem is cut and then the cut blood vessels are connected, which is a so-called end-to-end anastomosis method. At this time, if the cut blood vessel is too long, or when a long blood vessel is necessary in order to bypass the part of the blood vessel causing a problem, a leg vein is usually cut and used.

A cross section of a blood vessel is mainly comprised of an intima, a media, and an adventitia. When two blood vessels are anastomosed, the intima should come in close contact with another intima to be connected. When treatment of the above cardiovascular occlusive disease, a reconstructive surgery by a flap transfer surgery, or the anastomosis of the cut blood vessel is performed, since a method in which a microsurgery specialist directly uses a suture, secures a surgical field using a microscope or a high-power magnifying glass, and manually stitches one by one is used, such surgical suturing can be performed only by a highly skilled specialist and requires a great amount of time and effort. In particular, manually stitching and anastomosing blood vessels around a region that continuously and periodically beats such as a heart is very difficult. Accordingly, during cardiac surgery, a procedure in which heart attack is artificially induced to stop a heartbeat for at least three hours is necessary.

Therefore, in order to avoid manually and directly stitching the blood vessel using the suture, several vascular anastomosis devices have been designed. Among them, as a device for facilitating the end-to-end anastomosis of the blood vessel, U.S. Pat. No. 3,774,615, U.S. Pat. No. 4,214,586, and U.S. Pat. No. 4,917,087, and the like are disclosed. A microvascular anastomosis coupler (Synovis Micro Companies Alliance Inc. United States) has already been commercialized.

In U.S. Pat. No. 3,774,615 (Drahoslav Lim, et al.), a device for connecting cut blood vessels without an operation is disclosed. However, the anastomosis is not properly performed and blood is likely to leak in that device because blood vessels are not completely fixed at a region in which the blood vessels are anastomosed, it is not easy to uniformly alternately bond the vicinities of two cut blood vessels, and the region in which the cut parts meet is too small.

In U.S. Pat. No. 4,214,586 (Robert W. Mericle), a device that has a basic principle similar to that of U.S. Pat. No. 3,774,615, but firmly fixes tail ends of a cut blood vessel, is disclosed. However, the problem of the anastomosis not being properly performed since the region in which the cut parts of the blood vessel meet is too small has not been resolved.

In U.S. Pat. No. 4,917,087 (David J. Walsh, et al.), a vascular anastomosis device having a fixed tubular shape is disclosed. However, this device has a problem in that it can be used for an end-to-end or end-to-side anastomosis only when the diameters of two blood vessels are the same. The device is also inefficient because after intimae are anastomosed, the intima tends to be restored to its original state since the device lacks strength to maintain the intimae shape. Accordingly, although several vascular anastomosis devices have been disclosed as described above, vascular anastomosis is still performed by a specialist in a direct stitching manner.

Under these circumstances, the inventors have tried to address problems in that, during microscopic surgery on a very fine blood vessel, a procedure of reconstructing the blood vessel that has lost its shape due to loss of blood is very difficult and time consuming, the diameter decreases after the procedure was completed, and the like, which are unsatisfactory outcomes in the surgery. Therefore, the inventors have manufactured a vascular anastomosis device of a predetermined shape using a material or a body which has self-expandability in response to an external stimulus and is removable after a procedure, and have confirmed that the vascular anastomosis can be easily performed using the same, thereby completing the invention.

The present invention provides a vascular anastomosis device that comprises a cylindrical body compring a self-expandable material.

The present invention also provides a vascular anastomosis device that comprises a self-expandable wire and a thread connected to the self-expandable wire.

The present invention also provides a vascular anastomosis device that comprises an injection tube into which an expandable material is injected and a pair of elastic members are extended from one end of the injection tube.

The present invention also provides a vascular anastomosis device that has a cylindroid body and has outer diameter expandability.

The present invention also provides a vascular anastomosis device that comprises a self-expandable polymer ring and a thread connected to the self-expandable ring.

The present invention also provides a method of performing a vascular anastomosis using the vascular anastomosis device.

### SUMMARY OF THE INVENTION

According to an aspect of the present invention, there is provided a vascular anastomosis device comprising a cylindrical body having a diameter of 0.5 to 5 mm and a length of 10 to 60 mm, wherein the body comprises a material having self-expandability.

The term “vascular anastomosis device” used in the present invention refers to a body that fixes a blood vessel to easily perform a vascular anastomosis procedure or expands blood vessel diameters of both ends of a cut blood vessel.

In the present invention, the vascular anastomosis device having a cylindrical body is disposed between both ends of a cut blood vessel so as to be located at a lumen of the blood vessel, is radially self-expanded by an expanding agent applied from the outside to fix the blood vessel, and is removable by treating a solvent capable of dissolving the vascular anastomosis device after the ends of the blood vessel are stitched together. That is, the vascular anastomosis device having a cylindrical body is self-expanded due to pressure to thereby fix the blood vessel and helps the vascular anastomosis procedure be easily performed.

The stitching of the blood vessel performed before the vascular anastomosis device is removed may be preferably performed across a half to an entire end of the blood vessel.

The term “expandability” used in the present invention refers to a property of self-expansion in response to a specific external stimulus.

The material having self-expandability may comprise a starch, but the material is not limited thereto.

The term “expanding agent” used in the present invention refers to an external stimulus material for providing expandability.

The expanding agent may comprise a saline, or saline solution, but the expanding agent is not limited thereto.

The solvent capable of dissolving the vascular anastomosis device may comprise a saline, but the solvent is not limited thereto.

The vascular anastomosis device located at a lumen of the blood vessel may be treated with the solvent capable of dissolving the vascular anastomosis device from the outside through a syringe after the vascular anastomosis is performed. When the solvent is treated, the vascular anastomosis device is dissolved and is removable from the stitched blood vessel.

In addition, the present invention provides a method of vascular anastomosis comprising the following steps.

1) Disposing a vascular anastomosis device, which comprises a cylindrical body having a diameter of 0.5 to 5 mm and a length of 10 to 60 mm, wherein the body comprises a material having self-expandability, between both ends of a cut blood vessel so as to be located at a lumen of the blood vessel;

2) inducing the vascular anastomosis device to be radially self-expanded by treating the disposed vascular anastomosis device with an expanding agent;

3) bringing both ends of the blood vessel together over the self-expanded vascular anastomosis device;

4) stitching both of the ends of the blood vessel in contact with each other; and

5) removing the vascular anastomosis device by treating the vascular anastomosis device located inside the stitched blood vessel with a solvent capable of dissolving the vascular anastomosis device.

The stitching of the ends of the blood vessel that is performed before the vascular anastomosis device is removed may be performed across a half to an entire end of the blood vessel.

The method may further comprise 6) finishing the stitching of both ends of the blood vessel when the stitching of the ends of the blood vessel is not performed entirely.

Step 1 is a step in which the vascular anastomosis device, which comprises a cylindrical body having a diameter of 0.5 to 5 mm and a length of 10 to 60 mm, wherein the body comprises a material having self-expandability, is disposed between both ends of a cut blood vessel so as to be located at a lumen of the blood vessel. The vascular anastomosis device, having a cylindrical body that is self-expanded to fix the blood vessel due to a pressure, helps the vascular anastomosis procedure to be easily performed and is disposed between both ends of a cut blood vessel so as to be located at a lumen of the blood vessel.

Step 2 is a step in which the disposed vascular anastomosis device is treated with an expanding agent such that the vascular anastomosis device is induced to be radially self-expanded. The vascular anastomosis device is radially self-expanded toward an inner wall of the blood vessel to fix the blood vessel.

Step 3 is a step in which both ends of the blood vessel are brought together in the self-expanded vascular anastomosis device. In order to stitch the ends of the blood vessel, both ends of the blood vessel are brought together in the self-expanded vascular anastomosis device.

Step 4 is a step in which both of the ends of the blood vessel in contact with each other are stitched together. The ends of the blood vessel are stitched by a general stitching method.

Step 5 is a step in which the vascular anastomosis device located inside the stitched blood vessel is treated with a solvent capable of dissolving the vascular anastomosis device to remove the vascular anastomosis device. The vascular anastomosis device is removed after the ends of the blood vessel are stitched together.

Step 6 is a step in which the stitching of both ends of the blood vessel is finished. When the stitching of the ends of the blood vessel is not performed entirely, a remaining unstitched region is stitched to finish a stitching procedure.

The material having self-expandability, the expanding agent, the solvent capable of dissolving the vascular anastomosis device, and the method of treating the solvent capable of dissolving the vascular anastomosis device are the same as those in the vascular anastomosis device.

According to another aspect of the present invention, there is provided a vascular anastomosis device comprising a self-expandable wire having a diameter of 0.5 to 5 mm and a length of 10 to 60 mm, and a thread connected to the self-expandable wire.

The term “self-expandable wire” used in the present invention refers to a body that is spontaneously expanded to match a diameter of a blood vessel and supports the blood vessel when it is disposed in the blood vessel.

In the present invention, the self-expandable wire is disposed between both ends of a cut blood vessel so as to be located at a lumen of the blood vessel, is radially extended to fix the blood vessel, and is removable by pulling the thread connected to the wire from the outside after the ends of the blood vessel are stitched together. That is, the vascular anastomosis device may fix the blood vessel due to pressure according to an expansion of the wire, which may help the vascular anastomosis procedure be easily performed.

The stitching of the blood vessel performed before the vascular anastomosis device is removed may be preferably performed across a half to an entire end of the blood vessel.

A material of the self-expandable wire may be selected from among materials causing no vascular damage, and may specifically comprise nitinol, but the material is not limited thereto.

A material of the thread may comprise nitinol, but the material is not limited thereto.

In addition, the present invention provides a method of vascular anastomosis comprising the following steps.

1) In a vascular anastomosis device, which comprises a self-expandable wire having a diameter of 0.5 to 5 mm and a length of 10 to 60 mm, and a thread connected to the self-expandable wire, disposing the self-expandable wire between both ends of a cut blood vessel so as to be located at a lumen of the blood vessel, and disposing the thread connected to the self-expandable wire to be located outside the blood vessel;

2) inducing the disposed self-expandable wire to be radially extended;

3) bringing both ends of the blood vessel together in the expanded wire;

4) stitching both of the ends of the blood vessel in contact with each other; and

5) removing the wire by pulling the thread located outside the stitched blood vessel from the outside.

The stitching of the ends of the blood vessel performed before the expandable wire is removed may be performed across a half to an entire end of the blood vessel.

The method may further comprise 6) finishing the stitching of both ends of the blood vessel when the stitching of the ends of the blood vessel is not performed entirely.

Step 1 is a step in which, in a vascular anastomosis device, which comprises a self-expandable wire having a diameter of 0.5 to 5 mm and a length of 10 to 60 mm and a thread connected to the self-expandable wire, the self-expandable wire is disposed between both ends of a cut blood vessel so as to be located at a lumen of the blood vessel, and the thread connected to the self-expandable wire is disposed outside the blood vessel. The self-expandable wire of a predetermined size is disposed between both ends of the cut blood vessel so as to be located at the lumen of the blood vessel and the thread connected to the self-expandable wire is disposed so as to be located outside the blood vessel.

Step 2 is a step in which the disposed self-expandable wire is induced to be radially extended. The self-expandable wire is radially extended such that the lumen of the blood vessel is maximally expanded to fix the blood vessel due to a pressure.

Step 3 is a step in which both of the ends of the blood vessel are brought together in the expanded wire. In order to stitch the ends of the blood vessel, both ends of the blood vessel are brought together in the expanded wire.

Step 4 is a step in which both of the ends of the blood vessel in contact with each other are stitched together. The ends of the blood vessel are stitched together by a general stitching method.

Step 5 is a step in which the wire is removed by pulling the thread located outside the stitched blood vessel from the outside. After the ends of the blood vessel are stitched together, the thread connected to the wire is pulled to remove the wire. A pulled wire mesh is removed via a path through a pre-disposed thread without vascular damage. At this time, in order to prevent the vascular damage due to the pulled wire mesh, a body of the wire is formed such that it is narrowed and shrinks through an inside of a cylinder connected to the outside and a surface of the wire is coated in order to prevent the vascular damage.

Step 6 is a step in which the stitching of both ends of the blood vessel is finished. When the stitching of the ends of the blood vessel is not performed entirely, a remaining unstitched region is stitched to finish a stitching procedure.

Materials of the self-expandable wire and the thread are the same as those in the vascular anastomosis device.

According to still another aspect of the present invention, there is provided a vascular anastomosis device, comprising an injection tube into which an expandable material is injected, and a pair of elastic members extended from one end of the injection tube.

In the vascular anastomosis device, when the vascular anastomosis is performed, an expandable material is filled in the pair of elastic members such that the pair of elastic members are expanded to fix the blood vessel.

That is, in the present invention, the pair of elastic members are disposed between both ends of a cut blood vessel so as to be located at a lumen of the blood vessel, and are radially expanded by an expandable material injected through the injection tube to fix the blood vessel. After the ends of the blood vessel are stitched together, the expandable material filled in the pair of elastic members is removed from inside the pair of elastic members by the injection tube, and the injection tube is removable by being pulled from outside. That is, by filling the expandable material in the elastic member for expansion to fix the blood vessel, the vascular anastomosis device may help the vascular anastomosis procedure to be easily performed.

The stitching of the blood vessel performed before the vascular anastomosis device is removed may be preferably performed across a half to an entire end of the blood vessel.

The term “injection tube” used in the present invention refers to a tube having a path through which an expandable material is injected in the elastic member from the outside.

The term “elastic member” used in the present invention refers to a member having an expanding and shrinking property. Specifically, the elastic member is a member having a property that it may sufficiently expand as the expandable material filled therein expands, and shrink as the expandable material is removed.

A material of the elastic member may comprise a synthetic rubber, polyethylene, and combinations thereof, but the material is not limited thereto.

The vascular anastomosis device may further comprise a protrusion formed in the elastic member.

The term “protrusion” used in the present invention refers to a region having a sharply extruding or embossed shape.

The expandable material that can be used in the present invention may comprise a saline, air, or any combinations thereof, but the material is not limited thereto.

In addition, the present invention provides a method of vascular anastomosis comprising the following steps.

1) In a vascular anastomosis device comprising an injection tube into which an expandable material is injected and a pair of elastic members extended from one end of the injection tube, disposing the pair of elastic members between both ends of a cut blood vessel so as to be located at a lumen of the blood vessel, and disposing the injection tube to be located outside the blood vessel;

2) inducing the pair of elastic members to be radially extended by injecting the expandable material through the disposed injection tube of the vascular anastomosis device to fill the expandable material in the pair of elastic members;

3) bringing both ends of the blood vessel together in the pair of expanded elastic members;

4) stitching both of the ends of the blood vessel in contact with each other;

5) removing the expandable material filled in the pair of elastic members; and

6) removing the elastic member by pulling the injection tube from the outside.

The stitching of the ends of the blood vessel performed before the vascular anastomosis device is removed may be performed across a half to an entire end of the blood vessel.

The method may further comprise 7) finishing the stitching of both ends of the blood vessel when the stitching of the ends of the blood vessel is not performed entirely.

Step 1 is a step in which, in a vascular anastomosis device comprising an injection tube into which an expandable material is injected and a pair of elastic members extended from one end of the injection tube, the pair of elastic members are disposed between both ends of a cut blood vessel so as to be located at a lumen of the blood vessel, and the injection tube is disposed to be located outside the blood vessel. The pair of elastic members are disposed between both ends of the cut blood vessel so as to be located at the lumen of the blood vessel, and the injection tube into which an expandable material is injected is disposed to be located outside the blood vessel.

Step 2 is a step in which the pair of elastic members are induced to be radially extended by injecting the expandable material through the disposed injection tube of the vascular anastomosis device to fill the expandable material in the pair of elastic members. The expandable material is injected from the outside through the injection tube, the expandable material is filled in both elastic members, and the pair of elastic members are radially extended to fix the blood vessel.

Step 3 is a step in which both ends of the blood vessel are brought together in the pair of expanded elastic members. In order to stitch the ends of the blood vessel, both ends of the blood vessel are brought together in the pair of expanded elastic members.

Step 4 is a step in which both of the ends of the blood vessel in contact with each other are stitched together. The stitching of the ends of the blood vessel is performed by a general stitching method.

Step 5 is a step in which the expandable material filled in the pair of elastic members is removed. After the ends of the blood vessel are stitched together, the expandable material filled in the elastic member is removed.

Step 6 is a step in which the pair of elastic members from which the expandable material is removed are removed by pulling the injection tube from the outside. After the expandable material is removed, the shrunk elastic member is removed.

Step 7 is a step in which the stitching of both ends of the blood vessel is finished. When the stitching of the ends of the blood vessel is not performed entirely, a remaining unstitched region is stitched to finish a stitching procedure.

A material of the elastic member and the expandable material are the same as those in the vascular anastomosis device.

According to yet another aspect of the present invention, there is provided a vascular anastomosis device, comprising a cylindroid body having a major axis length of 3 to 10 mm, a minor axis length of 0.5 to 3 mm, and a height of 0.5 to 3 mm, wherein the cylindroid body comprises an expandable material.

In the present invention, the vascular anastomosis device having a cylindroid body is inserted into both ends of a cut blood vessel through an injection tube, disposed to be located at a lumen of the blood vessel, is expanded due to an expanding agent treated from the outside so as to expand a diameter of the blood vessel, and is removable to the outside during the last step of the stitching of the ends of the blood vessel. That is, the vascular anastomosis device having a cylindroid body is expanded in the blood vessel, and constantly maintains a lumen of the blood vessel due to a pressure, which may help the vascular anastomosis procedure to be easily performed.

The stitching of the blood vessel performed before the vascular anastomosis device is removed may be preferably performed across 70 to 85% of the ends of the blood vessel.

The term “expandability” used in the present invention refers to a property of self-expansion in response to a specific external stimulus.

The expandable material may comprise viscose rayon, but the material is not limited thereto. Preferably, the viscose rayon may have a compressed form.

The term “expanding agent” used in the present invention refers to an external stimulus material for providing expandability.

The expanding agent may comprise a saline, but the expanding agent is not limited thereto.

In a method of removing the vascular anastomosis device to the outside, a part of the body may be pulled from the outside to be removed, but the method is not limited thereto.

In addition, the present invention provides a method of vascular anastomosis comprising the following steps.

1) Inserting an injection tube having a cylindrical body with an inner diameter of 0.5 to 3 mm and a length of 20 to 40 mm into both ends of a cut blood vessel;

2) disposing a vascular anastomosis device, which comprises a cylindroid body having a major axis length of 3 to 10 mm, a minor axis length of 0.5 to 3 mm, and a height of 0.5 to 3 mm, wherein the cylindroid body comprises an expandable material, between both of the ends of the cut blood vessel so as to be located at a lumen of the blood vessel through the injection tube having a cylindrical body of step 1;

3) inducing the vascular anastomosis device to be expanded by treating the disposed vascular anastomosis device with an expanding agent;

4) bringing both ends of the blood vessel together over the expanded vascular anastomosis device;

5) stitching 70 to 85% of both of the ends of the blood vessel in contact with each other;

6) removing the vascular anastomosis device by pulling a specific part of the vascular anastomosis device located inside the stitched blood vessel to the outside of the blood vessel; and

7) stitching remaining unstitched ends of the cut blood vessel together after the vascular anastomosis device is removed.

The stitching of the ends of the blood vessel performed before the vascular anastomosis device is removed may be performed across 70 to 85% of the ends of the blood vessel.

Step 1 is a step in which an injection tube having a cylindrical body with an inner diameter of 0.5 to 3 mm and a length of 20 to 40 mm is inserted into both ends of a cut blood vessel. The injection tube which can make the vascular anastomosis device having a cylindroid body located in the blood vessel and which has a cylindrical body with an inner diameter of 1 to 3 mm and a length of 20 to 40 mm is located inside the blood vessel such that the vascular anastomosis device is located between both ends of the cut blood vessel so as to be located at the lumen of the blood vessel.

The term “injection tube” used in the present invention refers to a tube that secures a path through which the vascular anastomosis device is inserted into the blood vessel from the outside, and suppresses extension or expansion of a body having self-expandability or expandability to be extended or expanded at a desired location.

A material of the injection tube may comprise a polymer, and preferably, a synthetic polymer having biocompatibility, but the material is not limited thereto.

Step 2 is a step in which a vascular anastomosis device, which comprises a cylindroid body having a major axis length of 3 to 10 mm, a minor axis length of 0.5 to 3 mm, and a height of 0.5 to 3 mm, wherein the cylindroid body comprises an expandable material, is disposed between both ends of the cut blood vessel so as to be located at a lumen of the blood vessel through the injection tube having a cylindrical body of step 1. The vascular anastomosis device having a cylindroid body of a predetermined size is disposed to be located at a lumen of the end of the cut blood vessel through the disposed injection tube.

Step 3 is a step in which the vascular anastomosis device is induced to be expanded by treating the disposed vascular anastomosis device with an expanding agent. In order to fix the lumen of the blood vessel, the vascular anastomosis device is expanded such that a pressure applied onto an inner wall of the blood vessel appears.

Step 4 is a step in which both ends of the blood vessel are brought together over the expanded vascular anastomosis device. In order to stitch the ends of the blood vessel together, both ends of the blood vessel are brought together over the expanded vascular anastomosis device.

Step 5 is a step in which 70 to 85% of both of the ends of the blood vessel in contact with each other are stitched together. A predetermined part of both of the ends of the blood vessel in contact with each other is stitched.

Step 6 is a step in which the vascular anastomosis device is removed by pulling a specific part of the vascular anastomosis device located inside the stitched blood vessel to the outside of the blood vessel. A specific part of the vascular anastomosis device located in the stitched 70 to 85% of the blood vessel is pulled to the outside of the blood vessel and the vascular anastomosis device is removed.

Step 7 is a step in which remaining unstitched ends of the cut blood vessel are stitched together after the vascular anastomosis device is removed. The stitching of both ends of the blood vessel is finished.

The injection tube enabling the expandable material to be located inside the blood vessel, the expandable material, and the expanding agent are the same as those in the vascular anastomosis device.

According to yet another aspect of the present invention, there is provided a vascular anastomosis device, comprising a self-expandable ring having a diameter of 0.5 to 3 mm and a length of 0.7 to 1.7 mm, and a thread connected to the self-expandable ring.

The term “self-expandable ring” used in the present invention refers to a body that is spontaneously expanded to match a diameter of a blood vessel and supports the blood vessel when it is disposed in the blood vessel.

In the present invention, the self-expandable ring is disposed between both ends of a cut blood vessel so as to be located at a lumen of the blood vessel, is radially extended to fix the blood vessel, and is removable by pulling the thread connected to the ring from the outside after the ends of the blood vessel are stitched together. That is, the vascular anastomosis device fixes the blood vessel due to a pressure caused by expanding of the ring, which may help the vascular anastomosis procedure to be easily performed.

The stitching of the blood vessel performed before the vascular anastomosis device is removed may be preferably performed across 70 to 85% of the ends of the blood vessel.

A material of the self-expandable ring may be selected from among materials causing no vascular damage and having excellent biocompatibility, such as a polymer-based material, and specifically, may comprise PLLA (poly(L-lactic acid)), PLGA (poly(D,L-lactic-co-glycolic acid)), PMMA (poly methyl methacrylate), PHEMA (poly hydroxy ethyl methacrylate), PU (polyurethane), PE (polyethylene), or any combinations thereof, but the material is not limited thereto.

A material of the thread may comprise a synthetic polymer, but the material is not limited thereto. Specifically, the material of the thread may comprise Prolene (Ethicon, USA) but the material is not limited thereto.

In addition, the present invention provides a method of vascular anastomosis comprising the following steps.

1) Inserting an injection tube having a cylindrical body with an inner diameter of 0.5 to 3 mm and a length of 20 to 40 mm into both ends of a cut blood vessel;

2) disposing a vascular anastomosis device comprising a self-expandable ring having a diameter of 0.5 to 3 mm and a length 0.7 to 1.7 mm, and a thread connected to the self-expandable ring between both ends of a cut blood vessel so as to be located at a lumen of the blood vessel and disposing the thread connected to the self-expandable ring to be located outside the blood vessel;

3) inducing the self-expandable ring to be radially extended at an appropriate location inside the blood vessel by removing the injection tube from the disposed vascular anastomosis device;

4) bringing both ends of the blood vessel together over the expanded ring;

5) stitching 70 to 85% of both ends of the blood vessel in contact with each other;

6) removing the ring to the outside of the blood vessel by pulling the thread located outside the stitched blood vessel from the outside; and

7) stitching remaining unstitched ends of the cut blood vessel together after the self-expandable ring is removed.

The stitching of the ends of the blood vessel performed before the self-expandable ring is removed may be performed across 70 to 85% of the ends of the blood vessel.

Step 1 is a step in which an injection tube having a cylindrical body with an inner diameter of 0.5 to 3 mm and a length of 20 to 40 mm is inserted into both ends of a cut blood vessel. The injection tube having a cylindrical body is disposed between both ends of a cut blood vessel so as to be located at a lumen of the blood vessel such that the body of a self-expandable ring is located therein.

The term “injection tube” used in the present invention refers to a tube that secures a path through which the vascular anastomosis device is inserted into the blood vessel from the outside, and suppresses extension or expansion of a body having self-expandability or expandability to be extended or expanded at a desired location.

A material of the injection tube may comprise a polymer, and preferably, a synthetic polymer having biocompatibility, but the material is not limited thereto.

Step 2 is a step in which a vascular anastomosis device, which comprises a self-expandable ring having a diameter of 0.5 to 3 mm and a length of 0.7 to 1.7 mm, and a thread connected to the self-expandable ring, is disposed between both ends of a cut blood vessel so as to be located at a lumen of the blood vessel and the thread connected to the self-expandable ring is disposed to be located outside the blood vessel. Through the disposed injection tube, in the vascular anastomosis device comprising the self-expandable ring and the thread connected to the self-expandable ring, the self-expandable ring is disposed between both ends of the cut blood vessel so as to be located at the lumen of the blood vessel such that the lumen is fixed and maintained, and the thread connected to the self-expandable ring is disposed to be located outside the blood vessel.

Step 3 is a step in which the self-expandable ring is induced to be radially extended at an appropriate location inside the blood vessel by removing the injection tube from the disposed vascular anastomosis device. The injection tube, which suppresses expansion of the self-expandable ring, is removed to maximally expand the lumen of the blood vessel and the self-expandable ring is radially extended to fix the blood vessel due to a pressure.

Step 4 is a step in which both ends of the blood vessel are brought together over the expanded ring. In order to stitch the ends of the blood vessel together, both ends of the blood vessel are brought together over the expanded ring.

Step 5 is a step in which 70 to 85% of both ends of the blood vessel in contact with each other are stitched together. 70 to 85% of the ends of the blood vessel are stitched together by a general stitching method.

Step 6 is a step in which the ring is removed to the outside of the blood vessel by pulling the thread located outside the stitched blood vessel from the outside. After the ends of the blood vessel are partially stitched together, the ring is removed by pulling the thread connected to the ring.

In step 6, the pulled ring is removed through a path formed by a pre-disposed thread without vascular damage. At this time, in order to prevent vascular damage due to the pulled ring, a body of the ring spreads in a string shape in which one end is opened in a cylindrical shape, and an open end of the string is set to be rounded in order to prevent vascular damage.

Step 7 is a step in which remaining unstitched ends of the cut blood vessel are stitched together after the self-expandable ring is removed. The stitching of both ends of the blood vessel is finished.

Materials of the self-expandable ring and the thread are the same as those in the vascular anastomosis device.

According to yet another aspect of the present invention, there is provided a vascular anastomosis device which comprises a cylindrical body with an inner diameter of 0.5 to 5 mm and a length of 5 to 50 mm, and having a biodegradable body, an elastic member that is located adjacent to a lumen of the biodegradable body and radially expands the biodegradable body, and an injection tube which is a body extended from the elastic member and into which an expandable material is injected.

The term “biodegradability” used in the present invention refers to a material that can be decomposed in a living body within a predetermined time.

In the present invention, a material of the biodegradable body may comprise, for example, PLGA (poly(D,L-lactic-co-glycolic acid)), but the material is not limited thereto.

In the present invention, the biodegradable body is disposed between both ends of a cut blood vessel to be located at a lumen of the blood vessel, and is radially expanded due to the elastic member to fix the blood vessel. After the ends of the blood vessel are stitched together, when the biodegradable body remains inside the living body without change, it can be bio-degraded after a predetermined time. In addition, since the biodegradable body has an inner diameter of a predetermined size, even when it is located inside the blood vessel after the ends of the blood vessel are stitched together, a blood flow is not interfered with. That is, the biodegradable body is expanded with the aid of the elastic member, and fixes the blood vessel due to a pressure, which may help the vascular anastomosis procedure be easily performed.

The term “elastic member” used in the present invention refers to a body that is located adjacent to a lumen of the biodegradable body and radially expands the biodegradable body by the expandable material injected through the injection tube from the outside.

The term “elastic member” used in the present invention refers to a member having an expanding and shrinking property. Specifically, the elastic member is a member having a property that it may sufficiently expand as the expandable material filled therein expands, and shrink as the expandable material is removed.

A material of the elastic member may comprise a synthetic rubber, polyethylene, and combinations thereof, but the material is not limited thereto.

The term “injection tube” used in the present invention refers to a tube having a path through which an expandable material is injected in the elastic member from the outside.

The term “material having expandability” used in the present invention refers to a material that is filled inside the elastic member and expands the elastic member. Specifically, the expandable material that can be used in the present invention may comprise, for example, water, air, CO₂, or any combinations thereof, but the material is not limited thereto.

In addition, the present invention provides a method of vascular anastomosis comprising the following steps.

1) Disposing a vascular anastomosis device, which comprises a biodegradable body having a cylindrical body with an inner diameter of 0.5 to 5 mm and a length of 5 to 50 mm, an elastic member that is located adjacent to a lumen of the biodegradable body and radially expands the biodegradable body, and an injection tube which is a body extended from the elastic member and into which an expandable material is injected, between both ends of a cut blood vessel so as to be located at a lumen of the blood vessel and disposing the injection tube to be located outside the blood vessel;

2) inducing the biodegradable body to be expanded by injecting an expandable material through the disposed injection tube of the vascular anastomosis device, and filling the expandable material in the elastic member to induce the elastic member to be radially extended;

3) bringing both ends of the blood vessel together over the expanded biodegradable body;

4) stitching 70 to 85% of both of the ends of the blood vessel in contact with each other;

5) removing the expandable material in the elastic member through the injection tube located outside the stitched blood vessel and restoring the elastic member to a size of an initial state;

6) removing the elastic member to the outside of the blood vessel by pulling the injection tube located outside the stitched blood vessel from the outside; and

7) stitching remaining unstitched ends of the cut blood vessel together after the elastic member is removed.

The stitching of the ends of the blood vessel performed before the elastic member is removed may be performed across 70 to 85% of the ends of the blood vessel.

Step 1 is a step in which a vascular anastomosis device, which comprises a biodegradable body having a cylindrical body with an inner diameter of 0.5 to 5 mm and a length of 5 to 50 mm, an elastic member that is located adjacent to a lumen of the biodegradable body and radially expands the biodegradable body, and an injection tube which is a body extended from the elastic member and into which an expandable material is injected, is disposed between both ends of a cut blood vessel so as to be located at a lumen of the blood vessel and the injection tube is disposed to be located outside the blood vessel. The biodegradable body and the elastic member located adjacent to the lumen thereof are located inside the blood vessel.

Step 2 is a step in which the biodegradable body is induced to be expanded by injecting an expandable material through the disposed injection tube of the vascular anastomosis device, and filling the expandable material in the elastic member to induce the elastic member to be radially extended. The expandable material is filled in the elastic member to expand the elastic member. As a result, the biodegradable body is expanded.

Step 3 is a step in which both ends of the blood vessel are brought together over the expanded biodegradable body. In order to stitch the ends of the blood vessel together, both ends of the blood vessel are brought together over the expanded biodegradable body.

Step 4 is a step in which 70 to 85% of both of the ends of the blood vessel in contact with each other are stitched together. 70 to 85% of the ends of the blood vessel are stitched together by a general stitching method.

Step 5 is a step in which the expandable material in the elastic member is removed through the injection tube located outside the stitched blood vessel and the elastic member was restored to a size of an initial state. In order to easily remove the elastic member, the expandable material in the elastic member is removed.

Step 6 is a step in which the elastic member is removed to the outside of the blood vessel by pulling the injection tube located outside the stitched blood vessel from the outside. After the ends of the blood vessel are partially stitched together, the injection tube connected to the elastic member is pulled to remove the elastic member.

Step 7 is a step in which remaining unstitched ends of the cut blood vessel are stitched together after the elastic member is removed. The stitching of the ends of the blood vessel is finished.

A material of the biodegradable body, a material of the elastic member, and the expandable material are the same as those in the vascular anastomosis device.

The present invention may provide a vascular anastomosis device of a predetermined shape using a material or a body which has self-expandability in response to an external stimulus and is removable after a procedure. After the vascular anastomosis device is disposed between both ends of a cut blood vessel so as to be located at a lumen of the blood vessel, a predetermined external stimulus is applied to extend or expand the vascular anastomosis device and the blood vessel is fixed. As a result, it facilitates stitching the ends of the blood vessel together easily and performing the vascular anastomosis procedure easily.

### BRIEF DESCRIPTION OF THE DRAWINGS

FIG. 1 is a schematic diagram illustrating a vascular anastomosis device according to an embodiment of the present invention and a procedure of performing a vascular anastomosis using the same.

FIG. 2 is a schematic diagram illustrating a vascular anastomosis device according to another embodiment of the present invention and a procedure of performing a vascular anastomosis using the same.

FIG. 3 is a schematic diagram illustrating a vascular anastomosis device according to still another embodiment of the present invention and a procedure of performing a vascular anastomosis using the same.

FIG. 4 is a schematic diagram illustrating a vascular anastomosis device according to an embodiment of the present invention and a procedure of performing a vascular anastomosis using the same.

FIG. 5 is a schematic diagram illustrating a vascular anastomosis device according to an embodiment of the present invention and a procedure of performing a vascular anastomosis using the same.

FIG. 6 is a schematic diagram illustrating a vascular anastomosis device according to an embodiment of the present invention and a procedure of performing a vascular anastomosis using the same.

### DETAILED DESCRIPTION OF INVENTION

Hereinafter, the present invention will be described in detail with reference to following examples. However, these examples are for illustrative purpose only, and the scope of the present invention is not limited thereto.

### Example 1

### Vascular Anastomosis Using a Vascular Anastomosis Device of the Present Invention

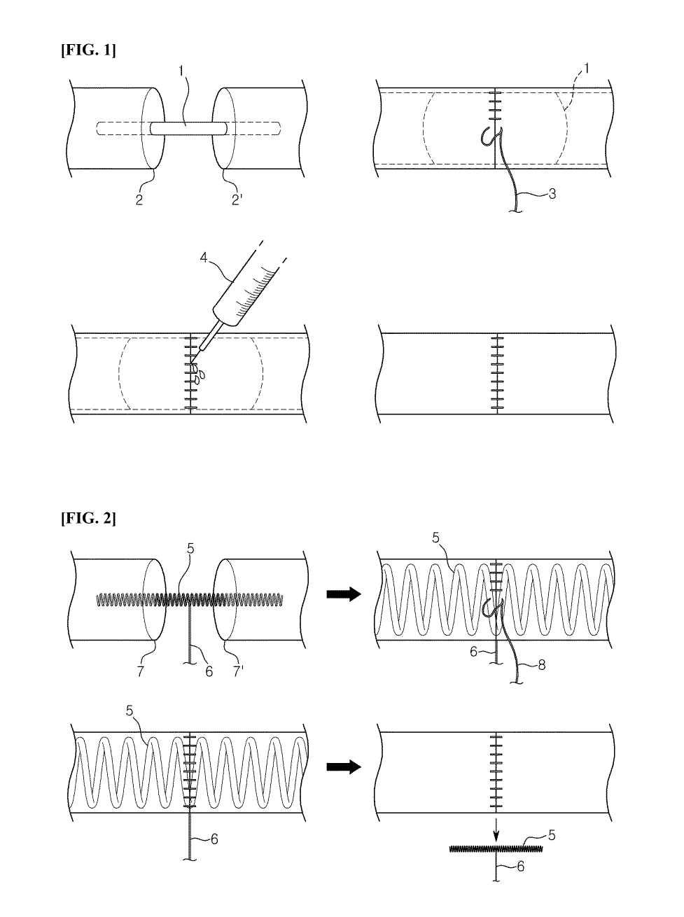

A vascular anastomosis was performed using a vascular anastomosis device of the present invention by a method illustrated in FIG. 1.

First, a vascular anastomosis device 1, which is a cylindrical body having a diameter of 5 mm and a length of 20 mm and comprises a material having self-expandability, was disposed between both ends 2 and 2′ of a cut blood vessel (a lumen size of 5 mm, rat femoral artery), so as to be located at a lumen of the blood vessel. The disposed vascular anastomosis device 1 was treated with a starch cylindrical body (a diameter of 2 mm and a length of 20 mm) as an expanding agent, and the vascular anastomosis device was induced to be radially self-expanded.

When self-expansion was completed up to a maximum of the lumen of the blood vessel, both ends 2 and 2′ were brought together over the self-expanded vascular anastomosis device 1. Then, both of the ends of the blood vessel in contact with each other were stitched together using Prolene (Ethicon, USA) as a suture 3.

After the stitching was completed, the vascular anastomosis device located inside the stitched blood vessel was treated with 10 ml of a saline as a solvent capable of dissolving the vascular anastomosis device through a syringe 4 to remove the vascular anastomosis device, and the vascular anastomosis was completed.

### Example 2

### Vascular Anastomosis Using a Vascular Anastomosis Device of the Present Invention

A vascular anastomosis was performed using a vascular anastomosis device of the present invention by a method illustrated in FIG. 2.

In a vascular anastomosis device comprising a self-expandable wire 5 having a diameter of 3 mm and a length of 20 mm and a thread 6 connected to the self-expandable wire, the self-expandable wire 5 was disposed between both ends 7 and 7′ of a cut blood vessel (a lumen size of 5 mm, rat femoral artery), so as to be located at a lumen of the blood vessel, and the thread 6 connected to the self-expandable wire 5 was disposed to be located outside the blood vessel. The disposed self-expandable wire 5 was induced to be radially extended.

When expanding was completed up to a maximum of the lumen of the blood vessel, both ends 7 and 7′ of the blood vessel were brought together over the self-expanded wire 5. Then, both of the ends 7 and 7′ of the blood vessel in contact with each other were stitched together using Prolene (Ethicon, USA) as a suture 8.

After the stitching was performed up to 70% of entire ends of the blood vessel, the thread 6 located outside the stitched blood vessel was pulled from the outside to remove the wire 5.

Then, the stitching of both ends 7 and 7′ of the blood vessel was finished, and the vascular anastomosis was completed.

### Example 3

### Vascular Anastomosis Using a Vascular Anastomosis Device of the Present Invention

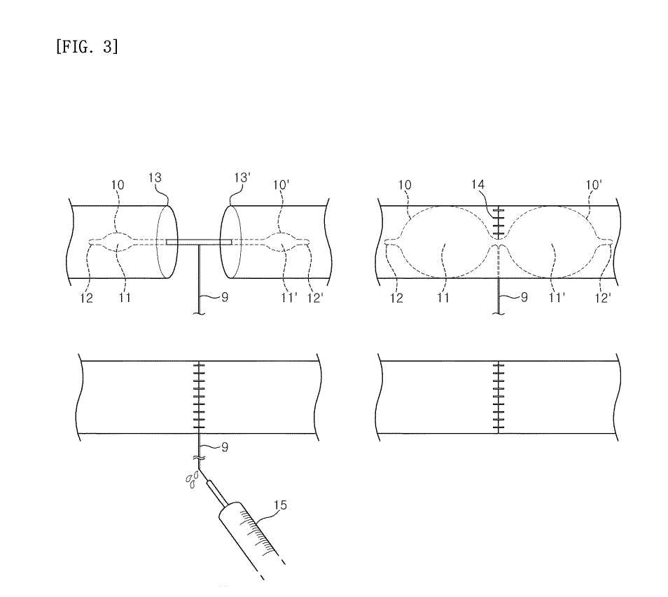

A vascular anastomosis was performed using a vascular anastomosis device of the present invention by a method illustrated in FIG. 3.

In a vascular anastomosis device comprising an injection tube 9 into which an expandable material was injected and a pair of elastic members 10 and 10′ extended from one end of the injection tube 9, the pair of elastic members 10 and 10′ were disposed between both ends 13 and 13′ of a cut blood vessel (a lumen size of 5 mm, rat femoral artery) so as to be located at a lumen of the blood vessel and the injection tube 9 was disposed outside the blood vessel. Through the disposed injection tube 9 of the vascular anastomosis device, a saline was injected as materials 11 and 11′ having expandability, insides of the pair of elastic members were filled with the saline serving as the materials 11 and 11′ having expandability, and the pair of elastic members 10 and 10′ were induced to be radially extended.

When expanding was completed up to a maximum of the lumen of the blood vessel, both ends 13 and 13′ of the blood vessel were brought together over the pair of expanded elastic members 10 and 10′. Then, both of the ends 13 and 13′ of the blood vessel in contact with each other were stitched together using Prolene (Ethicon, USA) as a suture 3.

After the stitching was completed, the materials 11 and 11′ having expandability filled inside the pair of elastic members were removed through the injection tube 9.

Then, the injection tube 9 was pulled from the outside to remove the pair of elastic members 10 and 10′ from which the materials 11 and 11′ having expandability were removed, and the vascular anastomosis was completed.

### Example 4

### Vascular Anastomosis Using a Vascular Anastomosis Device of the Present Invention

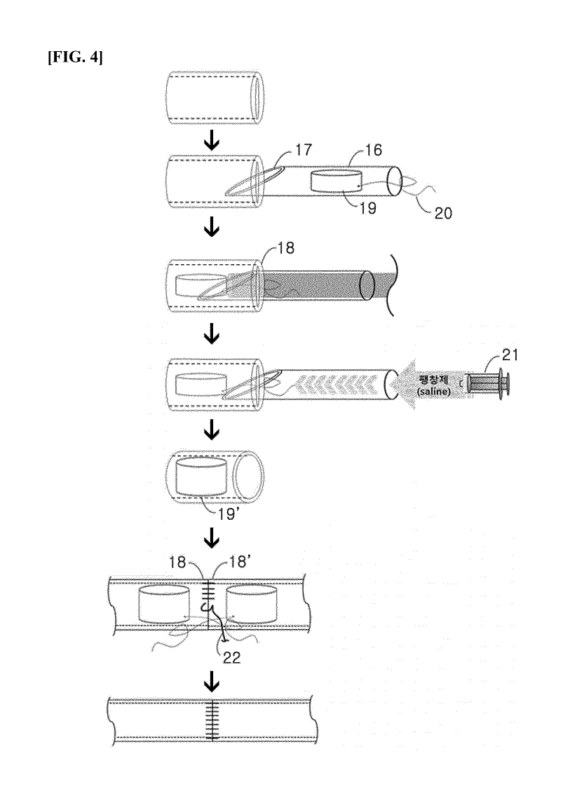

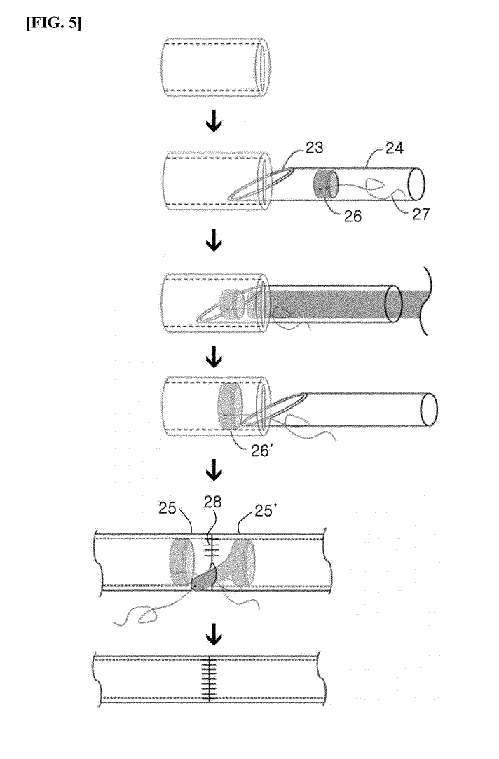

A vascular anastomosis was performed using a vascular anastomosis device of the present invention by a method illustrated in FIG. 4.

First, an injection tube 16, which had an outer diameter of 1.8 mm, an inner diameter of 1.2 mm, a length of 3.3 mm, and one oblique end 17, was inserted into a lumen of the blood vessel through both ends 18 and 18′ of a cut blood vessel. At this time, a vascular anastomosis device 19, which had a cylindroid body with a major axis length of 3 to 10 mm, a minor axis length of 0.5 to 3 mm, and a height of 0.5 to 3 mm, and comprised a material or a body having expandability, was located in the injection tube. Through one end of the injection tube 16 exposed to an outside of the blood vessel end, the cylindroid body 19 was pushed to be located inside the blood vessel using a pusher, 2 ml of a saline was treated as an expanding agent through a syringe 21, and the cylindroid body was induced to be expanded.

When expanding was completed up to a maximum of the lumen of the blood vessel, both ends 18 and 18′ of the blood vessel were brought together over an expanded vascular anastomosis device 19′. Then, both of the ends of the blood vessel in contact with each other were stitched together using Prolene (Ethicon, USA) as a suture 22 up to 75% of the ends.

Then, a thread 20 connected to the expanded vascular anastomosis device 19′ located inside the stitched blood vessel was pulled to remove the body, and the unstitched 25% of the ends was stitched together.

### Example 5

### Vascular Anastomosis Using a Vascular Anastomosis Device of the Present Invention

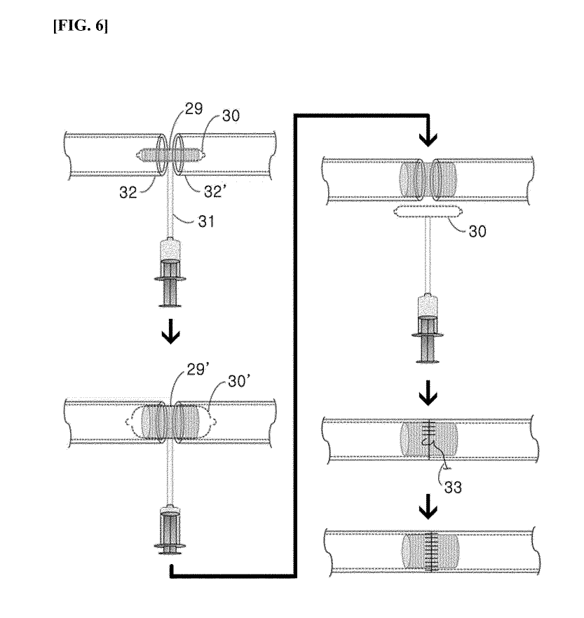

A vascular anastomosis was performed using a vascular anastomosis device of the present invention by a method illustrated in FIG. 5.

First, an injection tube 24, which had a diameter of 3 mm, a length of 3.3 mm, and one oblique end 23, was inserted into a lumen of the blood vessel through both ends 25 and 25′ of a cut blood vessel (a lumen size of 5 mm, pig femoral artery). A vascular anastomosis device comprising a self-expandable ring 26 having a diameter of 3 mm and a length of 1.7 mm and a thread 27 connected to the self-expandable ring was located inside the injection tube. Through one end of the injection tube 24 exposed to an outside of the blood vessel end, the self-expandable ring 26 was pushed to be located inside the blood vessel using a pusher. At this time, the thread 27 connected to the self-expandable ring was disposed to be located outside the blood vessel end. When the self-expandable ring 26 was moved from the injection tube 24 disposed inside the blood vessel, a pressure of the injection tube 24 that suppressed expansive force of the expandable ring 26 was released, and the self-expandable ring 26 was induced to be radially extended. When expansion was completed up to a maximum of the lumen of the blood vessel, both ends 25 and 25′ of the blood vessel were brought together over a self-expanded ring 26′. Then, both of the ends 25 and 25′ of the blood vessel in contact with each other were stitched together using Prolene (Ethicon, USA) as a suture 28. After the stitching was performed up to 85% of entire ends of the blood vessel, the thread 27 located outside the stitched blood vessel was pulled from the outside to remove the expanded ring 26′.

Then, the stitching of both ends 25 and 25′ of the blood vessel was finished and the vascular anastomosis was completed.

### Example 6

### Vascular Anastomosis Using a Vascular Anastomosis Device of the Present Invention

A vascular anastomosis was performed using a vascular anastomosis device of the present invention by a method illustrated in FIG. 6.

In both ends of a cut blood vessel, a vascular anastomosis device, which comprised a biodegradable body 29 having a cylindrical body with an inner diameter of 0.5 to 5 mm and a length of 5 to 50 mm, an elastic member 30 located adjacent to a lumen of the biodegradable body and radially expanded the biodegradable body, and an injection tube 31 which is a body extended from the elastic member and into which an expandable material is injected, was disposed between both ends 32 and 32′ of a cut blood vessel so as to be located at a lumen of the blood vessel and the injection tube was disposed to be located outside the blood vessel. Through the disposed injection tube of the vascular anastomosis device, air was injected as an expandable material to induce the elastic member to be radially extended, and the biodegradable body was induced to be expanded. Both ends 32 and 32′ of the blood vessel were brought together over the expanded biodegradable body 29′. Both ends of the blood vessel in contact with each other were stitched together up to 70 to 85%. Then, the air inside an expanded elastic member 30′ was removed through the injection tube 31 located outside the stitched blood vessel, and the elastic member was restored to a size of an initial state. The injection tube located outside the stitched blood vessel was pulled from the outside and the elastic member 30 was removed to the outside of the blood vessel. After the elastic member was removed, while the expanded biodegradable body 29′ was located at a lumen of the blood vessel, both ends of the blood vessel were stitched together using a suture 33, and the vascular anastomosis was completed.

1. A vascular anastomosis device, comprising
a cylindrical body having a diameter of 0.5 to 5 mm and a length of 10 to 60 mm,
wherein the body comprises a self-expandable material.

2. The device of claim 1,
wherein the device is disposed between both ends of a cut blood vessel so as to be located at a lumen of the blood vessel, is radially self-expanded by an expanding agent applied from the outside to fix the blood vessel, and is removable by treating a solvent capable of dissolving the device after the ends of the blood vessel are stitched together.

3. The device of claim 2,
wherein the stitching of the blood vessel performed before the device is removed is performed across a half to an entire end of the blood vessel.

4. The device of claim 1,
wherein the self-expandable material comprises starch.

5. The device of claim 2,
wherein the expanding agent comprises saline.

6. The device of claim 2,
wherein the solvent capable of dissolving the device comprises saline.

7. A vascular anastomosis device, comprising:
a self-expandable wire having a diameter of 0.5 to 5 mm and a length of 10 to 60 mm; and
a thread connected to the self-expandable wire.

8. The device of claim 7,
wherein the self-expandable wire is disposed between both ends of a cut blood vessel so as to be located at a lumen of the blood vessel, is radially extended to fix the blood vessel, and is removable by pulling the thread connected to the wire from the outside after the ends of the blood vessel are stitched together.

9. The device of claim 8,
wherein the stitching of the blood vessel performed before the self-expandable wire is removed is performed across a half to an entire end of the blood vessel.

10. The device of claim 7,
wherein a material of the self-expandable wire comprises nitinol.

11. The device of claim 7,
wherein a material of the thread comprises nitinol.

12. A vascular anastomosis device, comprising:
an injection tube into which an expandable material is injected; and
a pair of elastic members extended from one end of the injection tube.

13. The device of claim 12,
wherein a pair of the elastic members are disposed between both ends of a cut blood vessel so as to be located at a lumen of the blood vessel, are radially expanded by an expandable material injected through the injection tube to fix the blood vessel, and after the ends of the blood vessel are stitched together, the expandable material filled into the pair of the elastic members is removed from the inside of the pair of the elastic members through the injection tube, and the device is removed by pulling the injection tube from the outside.

14. The device of claim 13,
wherein the stitching performed before the device is removed is performed across a half to an entire end of the blood vessel.

15. The device of claim 12,
wherein a material of the elastic members comprises a synthetic rubber, polyethylene, or any combinations thereof.

16. The device of claim 12,
wherein the expandable material comprises saline, air, or any combinations thereof.

17. A vascular anastomosis device, comprising a cylindroid body having a major axis length of 3 to 10 mm, a minor axis length of 0.5 to 3 mm, and a height of 0.5 to 3 mm,
wherein the cylindroid body comprises an expandable material.

18. The device of claim 17,
wherein the expandable material comprises viscose rayon.

19. The device of claim 17,
wherein the expandable material is expanded by an expanding agent which comprises a saline.

20. A vascular anastomosis device, comprising:
a self-expandable ring having a diameter of 0.5 to 3 mm and a length of 0.7 to 1.7 mm; and
a thread connected to the self-expandable ring.

21. The device of claim 20,
wherein a material of the self-expandable ring comprises PLLA (poly(L-lactic acid)), PLGA (poly(D,L-lactic-co-glycolic acid)), PMMA (poly methyl methacrylate), PHEMA (poly hydroxy ethyl methacrylate), PU (polyurethane), PE (polyethylene), or any combinations thereof.

22. The device of claim 20,
wherein a material of the thread comprises Prolene.

23. A vascular anastomosis device, comprising:
a biodegradable body having a cylindrical body with an inner diameter of 0.5 to 5 mm and a length of 5 to 50 mm;
an elastic member that is located adjacent to a lumen of the biodegradable body and radially expands the biodegradable body; and
an injection tube which is a body extended from the elastic member and into which an expandable material is injected.

24. The device of claim 23,
wherein a material of the biodegradable body comprises PLGA (poly(D,L-lactic-co-glycolic acid)).

25. The device of claim 23,
wherein a material of the elastic member comprises a synthetic rubber, polyethylene, or any combinations thereof.

26. The device of claim 23,
wherein the expandable material comprises water, air, CO₂, or any combinations thereof.
